(19) United States
(12) Patent Application Publication
(10) Pub. No.: US 2024/0212202 A1
(43) Pub. Date: Jun. 27, 2024

(54) CENTERING DEVICE FOR DETERMINING A CENTERING OF A VISUAL AXIS OF AN EYE

(71) Applicant: SCHWIND EYE-TECH-SOLUTIONS GMBH, Kleinostheim (DE)

(72) Inventors: Samuel ARBA MOSQUERA, Aschaffenburg (DE); Mario SHRAIKI, Ober-Ramstadt (DE); Nico TRIEFENBACH, Mainaschaff (DE)

(21) Appl. No.: 18/391,131

(22) Filed: Dec. 20, 2023

(30) Foreign Application Priority Data
Dec. 22, 2022 (DE) 10 2022 134 615.3

(51) Int. Cl. G06T 7/73; A61B 3/12; A61F 9/008

(52) U.S. Cl. CPC G06T 7/73; A61B 3/12; A61F 9/00825; A61F 2009/00846; A61F 2009/00872; G06T 2207/10024; G06T 2207/30041

(57) ABSTRACT

The invention relates to a centering device (12) for determining a centering of a visual axis of an eye (14) to a beam path, wherein the centering device (12) comprises at least two color sources (16, 18), a control device (20) and a capturing device (22), wherein the color sources (16, 18) are arranged in the beam path of the centering device (12), wherein a respective color signal (17, 19) in a visible spectral range can be output by the color sources (16, 18) to an eye interface (26) via the beam path, wherein a wavelength of the respective color signals (17, 19) differs, and wherein the control device (20) is formed to control the capturing device (22) for ascertaining an eye orientation upon presence of a superposition criterion, by which a superposition of the color signals (17, 19) on the visual axis is indicated.

### FIELD

The invention relates to a centering device for determining a centering of a visual axis of an eye to a beam path, and to a treatment apparatus including at least one ophthalmological laser and such a centering device. Furthermore, the invention relates to a method for determining a centering of a visual axis of an eye, to a computer program for performing the method and to a computer-readable medium, on which the computer program is stored.

### BACKGROUND

In therapeutic and diagnostic systems in the ophthalmology, there is a challenge in centering an eye, in particular a visual axis of the eye, for a treatment and/or a diagnosis. Herein, the visual axis or axis of vision represents the viewing range of the sharpest vision of the eye, wherein a treatment or diagnosis shifted to the visual axis can result in undesired results. Therein, locating the correct centering of the eye based on the visual axis is difficult and time consuming.

Therefore, it is the object of the invention to improve a centering of a visual axis of an eye.

This object is solved by the independent claims. Advantageous embodiments of the invention are disclosed in the dependent claims, the following description as well as the figures.

### SUMMARY

The invention is based on the idea that a chromatic aberration is always present in the human eye, and thus different wavelengths, which are incident on the eye in decentered manner, are focused in the eye with different strength. Therefore, a transversal aberration arises in the eye, which can be used for centering. Herein, it is provided that two sources with different wavelengths are coaxially arranged in a beam path, wherein the visual axis is centered to the beam path if both wavelengths coincide in the retina.

By the invention, a centering device for determining a centering of a visual axis of an eye to a beam path is provided, wherein the centering device comprises at least two color sources, a control device and a capturing device, wherein the color sources are arranged in the beam path of the centering device, wherein a respective color signal in a visible spectral range can be output by the color sources to an eye interface via the beam path, wherein a wavelength of the respective color signals differs, and wherein the control device is formed to control the capturing device for ascertaining an eye orientation upon presence of a superposition criterion, by which a superposition of the color signals on the visual axis is indicated.

In other words, two different color signals are provided, which are together transferred in a beam path of the centering device, and can be output at an eye interface. Therein, an exit or window of the centering device is meant by an eye interface, at which an eye can be placed for examination and/or treatment. Thus, the eye interface is an interface between the centering device and the eye, which can for example comprise a contact element, onto which the eye can be pressed. By the eye interface, the color signals can enter the eye, which can be provided by color sources of the centering device. Therein, the color signals superimpose on each other in a position of the retina only if they are on the visual axis of the eye.

If this superposition is observed, the orientation of the eye is determined by means of a capturing device. For example, the capturing device can include a camera, which can take a picture of the eye with present superposition of the color signals on the visual axis since the visual axis is collinear to the beam path of the centering device upon superposition of the color signals. Otherwise, the color signals are apart from each other due to the chromatic aberration.

The color sources can include passive color sources, for example color points or color plates, which can preferably be illuminated with a light source, or active color sources, which generate a respective color signal, for example light sources, in particular laser light sources and/or color diodes.

The eye orientation can for example be ascertained by means of a picture of the eye and observation of landmarks, for example a pupil center, a corneal vertex, Purkinje images or characteristics of the iris in relation to the beam path of the centering device. The ascertained eye orientation with centered visual axis can be present in the form of data, which can be transferred to a diagnostic apparatus and/or a treatment apparatus. This means that the centering device can be used for therapeutic apparatuses such as for example ophthalmological lasers or diagnostic apparatuses, for example for an adaptation of glasses or contact lenses.

The superposition criterion, by which the superposition of the color signals on the visual axis is indicated, can be a rule or condition, which is examined to the effect whether or not the superposition of the color signals is present. For example, the superposition criterion can be present if the color signals are spatially one above the other on a retina, which can for example be optically ascertained, in particular by an ophthalmoscopy apparatus. Alternatively or additionally, an input device can also be provided, which a user or patient can use for input if the color signals are one above the other in a perception.

By the invention, the advantage arises that a centering of the eye can be performed in improved manner and in particular faster. Thus, improved diagnostic and/or treatment results can be achieved.

The invention also includes embodiments, by which additional advantages arise.

An embodiment provides that the color sources are formed as white light sources with respective color filters. This means that the color filters filter out respective wavelengths from the white light source and thus can provide a respectively different color signal.

A further embodiment provides that the color sources are formed as a light emitting diode and/or laser. Herein, collimated color signals can in particular be radiated by the respective color sources, whereby a superposition of the color sources can be better observed.

A further embodiment provides that the wavelengths of the color signals are at different ends of the visual spectral range, in particular one color signal in a red or orange spectral range and the other color signal in a blue or green spectral range. This means that the color signals, which are in the visible or visual spectral range, can differ in that one color signal has a wavelength, which is at a lower end of the spectral range, for example red or orange, and the wavelength of the other color signal can be at the upper end of the spectral range, for example blue or green. Thus, a suitable differentiation of the colors and/or an enhanced effect of the chromatic aberration can be provided, whereby an accuracy in the determination of the centering can be increased.

Preferably, it is provided that one of the color signals has a wavelength in the visible spectral range below 500 nanometers and/or the further color signal has a wavelength in the visual spectral range above 600 nanometers.

In a further advantageous embodiment, it is provided that the wavelengths of the color signals have a distance of at least 200 nanometers. This means that at least a wavelength difference of 200 nanometers is provided between the color signals.

A further embodiment provides that the capturing device is additionally formed to ascertain a superposition of the color signals on a retina, wherein the superposition criterion is present upon ascertainment of the superposition. In other words, the capturing device can for example also comprise an ophthalmoscopy apparatus in addition to a camera for capturing the eye orientation, by which a projection of the color signals on the retina can be captured. If it is observed in such captures that the color signals are one above the other on the retina, thus, it can be inferred that the visual axis of the eye is coaxial to the beam path of the centering device. Thus, the superposition criterion can be present and the associated eye orientation can be ascertained. By this embodiment, the advantage arises that an objective determination of the superposition can be provided.

A further embodiment provides that the capturing device is additionally formed to ascertain a superposition of Purkinje images of the color signals, wherein the superposition criterion is present upon ascertainment of the superposition. Reflections of the arriving color signals by optical interfaces of the eye are meant by Purkinje images, wherein four different Purkinje images can arise from four interfaces. Therein, one can arise on the surface of the cornea, one at the rear surface of the cornea, one on the front surface of the eye lens and one on the rear surface of the eye lens. For determining the superposition, the first Purkinje image and the fourth Purkinje image of the respective color signals can in particular be brought to superposition to determine the centering of the eye. In particular, not all of the Purkinje images of all of the color signals have to superimpose on each other in one point, but those of the first color signal respectively superimpose on each other in a first point and those of the second color signal in a second point. Hereby, a further objective determination of a centering of the eye can be assessed.

A further embodiment provides that the capturing device is formed to ascertain the eye orientation by means of a picture of the eye and a determination of landmarks in the picture, in particular of a pupil center and/or characteristics of the iris. Preferably, the landmarks can be captured in relation to the beam path of the centering device. This means that characteristic points of the eye or landmarks can be ascertained via a picture of the eye to ascertain the position and/or orientation of the eye, which are present upon the superposition of the color signals. These landmarks can then in particular be used in further steps, for example in a treatment of the eye, to recognize the centering or the visual axis.

A further embodiment provides that the centering device comprises an input device, which is formed for generating a control signal for the control device, wherein the superposition criterion is present by the generated control signal. In other words, by the input device, which can for example include a push button or switch, the patient can inform the centering device when he perceives the color signals one above the other, and thus manually trigger the superposition criterion. Hereby, further capturing devices can be omitted, which saves complexity and/or cost.

A further aspect of the invention relates to a treatment apparatus with at least one ophthalmological laser for the separation of a corneal volume of a human or animal eye by means of optical breakthrough, in particular by means of photodisruption and/or ablation, and/or for a laser induced structural change, in particular a laser induced refractive index change and/or laser induced cross-linking, and with the above mentioned centering device. Preferably, the beam path of the centering device and the beam path of the laser can be coaxial. The eye interface or patient interface of the centering device can also be the same as for the treatment apparatus, wherein the eye interface can preferably comprise a contact element for contacting the eye at the treatment apparatus or centering device. The respective laser can be formed to at least partially separate a predefined corneal volume with predefined interfaces of a human or animal eye by means of optical breakthrough, in particular at least partially separate it by means of photodisruption, and/or to ablate corneal layers by means of photo(ablation) and/or to effect a laser induced refractive index change in the cornea and/or the eye lens.

In an embodiment of the treatment apparatus, it is provided that the eye interface comprises a fixing device for the eye, in particular a suction ring, wherein the control device is additionally formed for controlling the fixing device for fixing the eye upon presence of the superposition criterion. This means that the eye can be fixed in the position, in which the color signals are one above the other on the visual axis, in that the fixing device fixes the eye. Thereto, the fixing device can for example be formed as a suction ring, which generates a negative pressure and thus retains the eye in position. Alternatively, it can also be examined by the capturing device when the eye orientation matches the ascertained eye orientation upon superposition of the color signals, wherein the fixing device can then be controlled for fixing the eye.

A further aspect of the invention relates to a method for determining a centering of a visual axis of an eye with the previously described centering device, wherein a respective color signal in a visible spectral range and with different wavelengths is generated by at least two color sources and output to an eye interface via an at least partially common beam path, wherein a presence of a superposition criterion, by which a superposition of the color signals on a visual axis of an eye located at the eye interface is indicated, is examined by a control device, wherein a capturing device ascertains an eye orientation if the superposition criterion is present. Herein, the same advantages and possibilities of variation as in the centering device arise. The respective method can include at least one additional step, which is executed if and only if an application case or an application situation occurs, which has not been explicitly described here. For example, the step can include the output of an error message and/or the output of a request for inputting a user feedback. Additionally or alternatively, it can be provided that a default setting and/or a predetermined initial state are adjusted.

According to the invention, a computer program is also provided, including commands, which cause the centering device to execute the above mentioned method. The computer program includes commands, which for example form a program code. Upon execution of the program code by means of a computer or a computer cluster, it is caused to execute the previously described method or at least one embodiment thereof.

A further aspect of the invention relates to a computer-readable medium (storage medium), on which the above mentioned computer program and the commands thereof, respectively, are stored. For executing the computer program, a computer or a computer cluster can access the computer-readable medium and read out the content thereof. The storage medium is for example formed as a data memory, in particular at least partially as a volatile or a non-volatile data memory. A non-volatile data memory can be a flash memory and/or an SSD (solid state drive) and/or a hard disk. A volatile data memory can be a RAM (random access memory). For example, the commands can be present as a source code of a programming language and/or as assembler and/or as a binary code.

A further aspect of the invention relates to a control device, which is formed to perform the steps of at least one embodiment of the previously described method. Thereto, the control device can comprise a computing unit for electronic data processing such as for example a processor. The computing unit can include at least one microcontroller and/or at least one microprocessor. The computing unit can be configured as an integrated circuit and/or microchip. Furthermore, the control device can include an (electronic) data memory or a storage unit. A program code can be stored on the data memory, by which the steps of the respective embodiment of the respective method are encoded. The program code can include the control data for the respective laser. The program code can be executed by means of the computing unit, whereby the control device is caused to execute the respective embodiment. The control device can be formed as a control chip or control unit. The control device can for example be encompassed by a computer or computer cluster.

Further features and advantages of one of the described aspects of the invention can result from the embodiments of another one of the aspects of the invention. Thus, the features of the embodiments of the invention can be present in any combination with each other if they have not been explicitly described as mutually exclusive.

### BRIEF DESCRIPTION OF THE DRAWINGS

In the following, additional features and advantages of the invention are described in the form of advantageous embodiments based on the figure(s). The features or feature combinations of the embodiments described in the following can be present in any combination with each other and/or the features of the embodiments. This means, the features of the embodiment can supplement and/or replace the features of the embodiments and vice versa. Thus, configurations are also to be regarded as encompassed and disclosed by the invention, which are not explicitly shown or explained in the figures, but arise from and can be generated by separated feature combinations from the embodiments. Thus, configurations are also to be regarded as disclosed, which do not comprise all of the features of an originally formulated claim or extend beyond or deviate from the feature combinations set forth in the relations of the claims.

FIG. 1 depicts a schematic representation of a centering device according to an exemplary embodiment.

FIG. 2 depicts a schematic representation of a treatment apparatus with a centering device according to an exemplary embodiment.

In the figures, identical or functionally identical elements are provided with the same reference characters.

### DETAILED DESCRIPTION

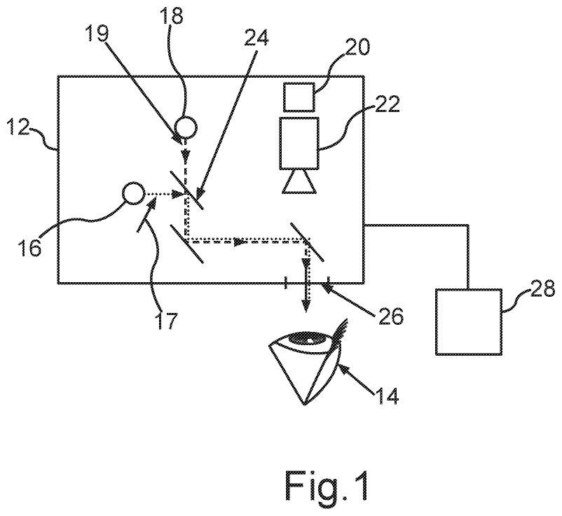

In FIG. 1, a schematically illustrated centering device 12 for determining a centering of a visual axis of an eye 14 according to an exemplary embodiment is illustrated. This means that the centering device 12 can be formed to center the visual axis of the eye, which specifies an area of sharpest vision, and to determine the associated orientation of the eye 14, respectively, such that the visual axis coaxially coincides with a beam path of a diagnostic and/or treatment apparatus, in particular if the beam path or beam exit of the diagnostic and/or treatment apparatus is in a neutral position. Thereto, the centering device 12 can comprise a first color source 16, a second color source 18, a control device 20 and a capturing device 22.

The first color source 16 can be formed to generate a first color signal 17. Thereto, the first color source 16 can for example be formed as a light emitting diode and/or laser. The color signal 17 generated by the first color source 16 can in particular be provided in a visible spectral range and preferably have a wavelength above 600 nanometers. In other words, the first color signal can for example be generated in a red or orange spectral range.

The second color source 18 can be formed to generate a second color signal 19, wherein the second color signal 19 has a wavelength different from the first color signal 17. The second color signal 19 can also be provided in the visible spectral range and preferably have a wavelength below 500 nanometers, wherein a wavelength difference of the color signals 17, 19 is preferably greater than 200 nanometers. This means that the second color signal 19 can for example be radiated in a blue or green spectral range.

Subsequently, the two color signals 17, 19 can be combined in a beam path of the centering device 12, for example by a mirror 24. Preferably, the mirror 24 can be formed as a partially transparent mirror and/or dichroitic mirror or filter.

Subsequently, the color signals 17, 19 can be passed to an eye interface 26 via the common beam path of the centering device 12, wherein the eye interface 26 provides an exit of the color signals 17, 19 to the outside and allows radiation into the eye 14. For example, a contact element can be provided at the eye interface 26, to which the eye 14 can be fitted.

In order to determine if a visual axis of the eye 14 is centered to the beam path of the respective color signals 17, 19, it can be examined if the color signals 17, 19 superimpose on each other on a visual axis of the eye 14. Due to a chromatic aberration, a refraction of different magnitude between the first color signal 17 and the second color signal 19 in the eye 14 is present upon decentration of the visual axis of the eye 14, whereby a transversal shift of the color signals 17, 19 occurs due to the decentration and thus the respective color signals 17, 19 impinge on different positions in the retina. However, if the common beam path of the color signals 17, 19 coaxially coincides with the visual axis or achromatic axis, the two color signals 17, 19 impinge on the retina in a common position. It is to be mentioned at this place that a chromatic aberration also occurs upon coincidence of the visual axis with the beam path. However, it only acts longitudinally to the visual axis/beam path, whereby a transversal shift is not present, such that the projections of the color signals 17, 19 on the retina overlap each other in a position.

In order to determine that the color signals 17, 19 are on the visual axis of the eye 14, the control device 20 can examine different superposition criteria, by which a superposition of the color signals on the visual axis is indicated. For example, a projection of the color signals 17, 19 on the retina can be captured by the capturing device 22, which can for example comprise an ophthalmoscopy apparatus. Subsequently, these captures can be examined by the control device 20 to the effect if the color signals 17, 19 are one above the other on the retina. If this is the case, the superposition criterion can be satisfied, whereby the control device 20 can control the capturing device 22 to ascertain an eye orientation. For example, the capturing device 22 can additionally include a camera, which takes a picture of the eye 14 at the moment when the superposition criterion is present. From this picture, for example based on landmarks, in particular based on a pupil center and/or characteristics of the iris, the orientation of the eye 14 in relation to the centering device 12 can then be ascertained, which corresponds to a centering of the visual axis.

A further possibility of examining the superposition criterion is ascertaining Purkinje images of the color signals 17, 19, which can for example also be captured by the capturing device 22. If the Purkinje images of the respective color signals 17, 19, in particular the first and the fourth Purkinje image of the respective color signals 17, 19, are one above the other, it can be provided that the control device 20 controls the capturing device 22 for ascertaining the eye orientation.

Alternatively or additionally, the centering device 12 can also include an input device 28, which can be manually operated by a user, in particular a patient. For example, the input device 28 can be formed as a button or switch. If the two color signals 17, 19 are situated one above the other in a perception of the patient, the patient can manually trigger the capture of the eye orientation via the input device 28.

The captured eye orientation can for example be stored in a storage device (not shown) of the centering device 12 and/or be transferred to further therapeutic and/or diagnostic apparatuses, which can use the eye orientation and thereby the centering of the visual axis of the eye 14 for further treatment and diagnostic steps.

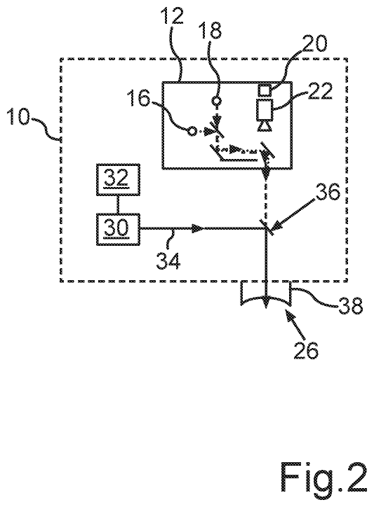

In FIG. 2, a schematically illustrated treatment apparatus 10 according to an exemplary embodiment is illustrated. The treatment apparatus 10 comprises an ophthalmological laser 30, for example for the correction of a cornea of an eye 14, wherein a correction profile, in particular a volume body or lenticule (not shown), can for example be defined by control data, which can be separated from the cornea by means of photodisruption and/or ablation. For example, interfaces can be preset in the control data for separating the lenticule, on which a cavitation bubble path for separating the lenticule from the cornea can be generated.

One recognizes that a control device 32 of the treatment apparatus 10 can be formed for the laser 30 besides the laser 30, such that it can emit pulsed laser pulses for example in a predefined pattern for generating the correction profile or the interfaces. Alternatively, the control device 32 of the treatment apparatus and the control device 20 of the centering device 12 can be formed as a common control unit.

Furthermore, FIG. 1 shows that the laser beam 34 generated by the laser 30 can be deflected to different positions by means of a beam device 36, namely a beam deflection device such as for example a rotation scanner, wherein a neutral position can preferably be defined, in which the laser beam 34 is radiated in centered or central manner. The beam deflection device 36 can also be controlled by the control device 32 to generate the correction profile or the interfaces.

Preferably, the illustrated laser 30 can be a photodisruptive and/or ablative laser, which is formed to emit laser pulses in a wavelength range between 300 nm and 1400 nm, preferably between 700 nm and 1200 nm, at a respective pulse duration between 1 fs and 1 ns, preferably between 10 fs and 10 ps, and a repetition frequency of greater than 10 kHz, preferably between 100 kHz and 100 MHz. In addition, the control device 32 optionally comprises a storage device (not illustrated) for at least temporary storage of at least one control dataset, wherein the control dataset or datasets include(s) control data for positioning and/or for focusing individual laser pulses in the cornea. The position data and/or the focusing data of the individual laser pulses, that is the correction profile of the lenticule to be separated, are generated based on predetermined control data, in particular from previously measured visual disorder data, in particular a previously measured topography and/or pachymetry and/or the morphology of the cornea.

Furthermore, the treatment apparatus 10 can comprise a centering device 12. Herein, it can be provided that the eye interface 26 of the centering device 12 is the same as that of the treatment apparatus 10. In particular, the eye interface 26 can comprise a fixing device 38, preferably a suction ring. The fixing device 38 can be formed to fix the eye 14 in a position and/or in an eye orientation.

Particularly preferably, the eye 14 can be docked to the fixing device 38 of the eye interface 26, wherein the centering device 12 examines the superposition of the color signals 17, 19. If the superposition of the color signals 17, 19 on the visual axis is present, the control device 20 and/or the control device 32 can additionally control the fixing device 38 for fixing the centered eye. This means that a suction device can for example be started, which sucks the eye by means of negative pressure and thus fixes it. Subsequently, the treatment of the centered eye 14 by means of the treatment apparatus 10 can be started.

Overall, the examples show how an automatic centering of a visual axis of an eye can be provided by the invention.

1. A centering device for determining a centering of a visual axis of an eye to a beam path, wherein the centering device comprises:
at least two color sources arranged in the beam path of the centering device, wherein the at least two color sources are configured to output a respective color signal in a visible spectral range to an eye interface via the beam path, wherein a wavelength of the respective color signals are different;
a capturing device; and
a control device configured to control the capturing device for ascertaining an eye orientation upon presence of a superposition criterion, the superposition criterion comprising a superposition of the color signals on the visual axis is indicated.

2. The centering device according to claim 1, wherein the at least two color sources comprise white light sources with respective color filters.

3. The centering device according to claim 1, wherein the at least two color sources comprise a light emitting diode and/or a laser.

4. The centering device according to claim 1, wherein the wavelengths of the color signals are at different ends of the visible spectral range.

5. The centering device according to claim 1, wherein one of the color signals has a wavelength in the visible spectral range below 500 nm and/or the another color signal of the color signals has a wavelength in the visual spectral range above 600 nm.

6. The centering device according to claim 1, wherein the wavelengths of the color signals are at least 200 nm.

7. The centering device according to claim 1, wherein the capturing device is further configured to ascertain a superposition of the color signals on a retina, wherein the superposition criterion is met upon ascertainment of the superposition.

8. The centering device according to claim 1, wherein the capturing device is further configured to ascertain a superposition of Purkinje images of the color signals, wherein the superposition criterion is met upon ascertainment of the superposition.

9. The centering device according to claim 1, wherein the capturing device is further configured to ascertain the eye orientation by means of a picture of the eye and a determination of landmarks in the picture.

10. The centering device according to claim 1, wherein the centering device comprises an input device configured to generate a control signal for the control device, wherein the superposition criterion is generated in response to the generated control signal.

11. A treatment apparatus with at least one ophthalmological laser for separation of a corneal volume of a human or animal eye by means of optical breakthrough, and/or for a laser induced structural change with the centering device according to claim 1.

12. The treatment apparatus according to claim 11, wherein the eye interface comprises a fixing device for the eye, wherein the control device is further configured to control the fixing device for fixing the eye in response to determining the superposition criterion is present.

13. A method for determining a centering of a visual axis of an eye via the centering device according to claim 1, the method comprising:
generating, via the at least two color sources, respective color signals in a visible spectral range and with different wavelengths;
outputting, via the at least two color sources, along an at least partially common beam path, the generated respective color signals to an eye interface;
determining, via the control device a presence of a superposition criterion based on a superposition of the color signals on a visual axis of an eye located at the eye interface; and
ascertaining, via the capturing device, an eye orientation in response to determining the superposition criterion is present.

14. (canceled)

15. A non-transitory computer-readable medium, on which a computer program is stored, the computer program comprising commands that cause the centering device to execute the method according to claim 13.

16. The centering device according to claim 4, wherein one color signal of the color signals corresponds to a red or orange spectral range and another color signal of the color signals corresponds to a blue or green spectral range.

17. The centering device according to claim 9, wherein, the landmarks comprise a pupil center and/or characteristics of an iris.

18. The treatment apparatus of claim 11, wherein the optical breakthrough comprises photodisruption and/or ablation; and
the laser induced structural change comprises laser induced refractive index change and/or laser induced cross-linking.

19. The treatment apparatus of claim 12, wherein the fixing device comprises a suction ring.
